(19) United States
(12) Patent Application Publication
(10) Pub. No.: US 2016/0143571 A1
(43) Pub. Date: May 26, 2016

(54) METHOD AND SYSTEM FOR DETERMINING PSYCHOLOGICAL DISORDER CONDITION IN A PERSON AND PROVIDING ASSISTANCE THEREFOR

(71) Applicant: Wipro Limited, Bangalore (IN)

(72) Inventors: Upendra Suddamalla, Anantapur Dt. (IN); Satish Prasad Rath, Thuberahally (IN)

(21) Appl. No.: 14/618,569

(22) Filed: Feb. 10, 2015

(30) Foreign Application Priority Data
Nov. 26, 2014 (IN) 5919/CHE/2014

(51) Int. Cl. A61B 5/16; A61B 5/0488; A61B 5/11

(52) U.S. Cl. CPC A61B 5/165; A61B 5/1114; A61B 5/0488

(57) ABSTRACT

This technology relates to a method and system for determining psychological disorder condition in a person and providing assistance to the person with the psychological disorder. One or more sensors are placed on the person to monitor the activities of the person. The one or more sensors provide sensor data associated with the monitored activities of the person to a computing unit. The computing unit detects the activities and identifies a recurring activity. For the recurring activity, the computing unit determines probability parameters and based on the probability parameters the probability of the psychological condition is determined. For providing assistance to the person with psychological disorder condition, the computing compares the activity with the disorder activities stored in the computing unit. If a match is found, then one or more indications are provided to the person to indicate that the activity has already been performed.

This application claims the benefit of Indian Patent Application No. 5919/CHE/2014 filed Nov. 26, 2014, which is hereby incorporated by reference in its entirety.

### FIELD

This technology is related, in general to monitoring health condition of a person, and more particularly, but not exclusively to a method and system for determining psychological disorder condition in a person and providing assistance for person with psychological disorder.

### BACKGROUND

The psychological disorder is a mental or behavioral pattern that causes impaired ability to function in ordinary life and which is not developmentally or socially normal. One such psychological disorder is Obsessive Compulsive Disorder (OCD). People with OCD feel the need to check things repeatedly or perform routines and rituals again and again. Different obsessions cause the person to carry out activities repetitively. For example, repeatedly checking if the door is locked, switching off lights if the lights are on, washing hands etc. OCD is accompanied by eating disorders, other anxiety disorders, or even depression.

OCD involves both obsessions and compulsions. The frequent upsetting thoughts are called obsessions. In order to control them, a person will feel an overwhelming urge to repeat certain rituals or behaviors called compulsions. People with OCD can't control these obsessions and compulsions. The symptoms may come and go, ease over time, or even get worse.

For treating the OCD condition, the different OCD symptoms which typically vary from person to person have to be identified. At present, there are very few techniques to identify the symptoms of OCD. The OCD is generally treated using cognitive-behavioral therapy, antidepressants etc. The problem with the cognitive-behavioral therapy is that people have to personally visit the psychiatrists many times for monitoring the symptoms of OCD. The psychiatrists will monitor the behavior of the person to identify the symptoms of OCD. The problem with using the antidepressants is that they are addictive and also may have side effects.

### SUMMARY

One or more shortcomings of the prior art are overcome and additional advantages are provided through the present disclosure. Additional features and advantages are realized through the techniques of the present disclosure. Other embodiments and aspects of the disclosure are described in detail herein and are considered a part of the claimed disclosure.

A method for determining psychological disorder condition in a person includes receiving sensor data at predefined time intervals from one or more sensors. Upon receiving the sensor data, the one or more activities performed by the person are identified. Thereafter, at least one recurring activity in the one or more activities is identified. The method further comprises determining one or more probability parameters for the at least one recurring activity. For each of the one or more probability parameters a predefined weighed ratio is assigned to generate one or more weighted probability parameters. Based on each of the one or more weighted probability parameters, the probability of the psychological disorder condition is determined.

A method for providing assistance to a person with psychological disorder condition includes receiving sensor data from one or more sensors. Based on the sensor data, the one or more activities performed by the person are identified. The method further comprises comparing the one or more activities with a list of one or more predefined disorder activities. Based on the comparison, one or more indications are provided in real-time for assisting the person with psychological disorder condition.

A computing unit that is configured to be capable of determining psychological disorder condition in a person includes a processor and a memory communicatively coupled to the processor. The memory stores processor executable instructions, which, on execution, cause the processor to receive sensor data at predefined time intervals from one or more sensors placed on the person. The instructions cause the processor to identify one or more activities performed by the person at each predefined time interval based on the sensor data. Thereafter, the at least one recurring activity is identified in the one or more activities. The instructions further cause the processor to determine one or more probability parameters for the at least one recurring activity. Thereafter, a predefined weighed ratio is assigned to each of the one or more probability parameters to generate one or more weighted probability parameters. Based on each of the one or more weighted probability parameters, the probability of the psychological disorder condition is determined.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The accompanying drawings, which are incorporated in and constitute a part of this disclosure, illustrate exemplary embodiments and, together with the description, serve to explain the disclosed principles. In the figures, the left-most digit(s) of a reference number identifies the figure in which the reference number first appears. The same numbers are used throughout the figures to reference like features and components. Some embodiments of system and/or methods in accordance with embodiments of the present subject matter are now described, by way of example only, and with reference to the accompanying figures, in which:

FIG. 1a illustrates an environment for determining psychological disorder condition of a person and for providing assistance to a person with psychological disorder in accordance with some embodiments of the present disclosure;

FIG. 1b illustrates a block diagram of an example of a psychological disorder management computing unit device or computing unit in accordance with some embodiments of the present disclosure;

FIG. 1c illustrates a detailed block diagram of a computing unit in accordance with some embodiments of the present disclosure;

FIG. 2 illustrates an exemplary environment for determining psychological disorder condition in a person in accordance with some embodiments of the present disclosure;

FIG. 3 illustrates an exemplary environment for providing assistance to person with psychological disorder in real-time in accordance with some embodiments of the present disclosure;

FIG. 4 shows a flowchart illustrating a method for determining psychological disorder condition in a person in accordance with some embodiments of the present disclosure;

FIG. 5 shows a flowchart illustrating a method for providing assistance to a person with psychological disorder in real-time in accordance with some embodiments of the present disclosure; and

FIG. 6 illustrates a block diagram of an example of a computing unit that is configured to be capable of implementing embodiments consistent with the present disclosure.

It should be appreciated by those skilled in the art that any block diagrams herein represent conceptual views of illustrative systems embodying the principles of the present subject matter. Similarly, it will be appreciated that any flow charts, flow diagrams and the like represent various processes which may be substantially represented in computer readable medium and executed by a computer or processor, whether or not such computer or processor is explicitly shown.

### DETAILED DESCRIPTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the particular forms disclosed, but on the contrary, the disclosure is to cover all modifications, equivalents, and alternative falling within the spirit and the scope of the disclosure.

The terms “comprises”, “comprising”, or any other variations thereof, are intended to cover a non-exclusive inclusion, such that a setup, device or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or apparatus.

Accordingly, the present disclosure relates to a method and a computing unit for determining psychological disorder condition in a person. The computing unit receives sensor data from one or more sensors. The one or more sensors are placed on the person. Using the sensor data, the computing unit identifies one or more activities of the person. The computing unit compares the one or more activities with each other to identify at least one recurring activity. The computing unit identifies one or more probability parameters for each recurring activity. For each of the one or more probability parameters, a predefined weighted ratio is assigned to generate a weighted probability parameter. The computing unit determines probability of the psychological disorder condition based on each of the weighted probability parameter.

In an embodiment, the present disclosure provides a method and the computing unit for assisting a person with psychological disorder condition in real-time. The computing unit receives sensor data from one or more sensors. Using the sensor data, the computing unit identifies one or more activities of the person. The computing unit stores a list of predefined disorder activities which the person feels are the symptoms of psychological disorder condition. If the one or more activities match with the predefined disorder activities, then the computing unit provides one or more indications in real-time indicating information about the activity being performed.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

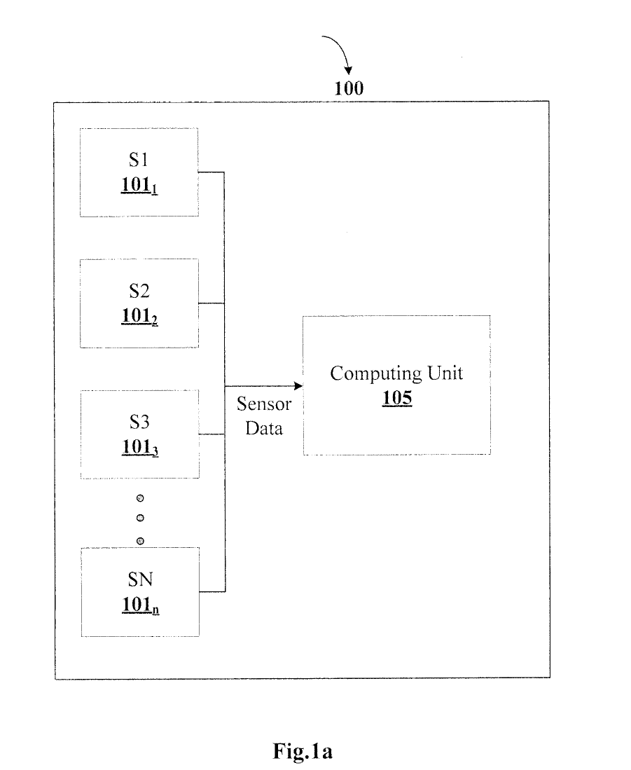

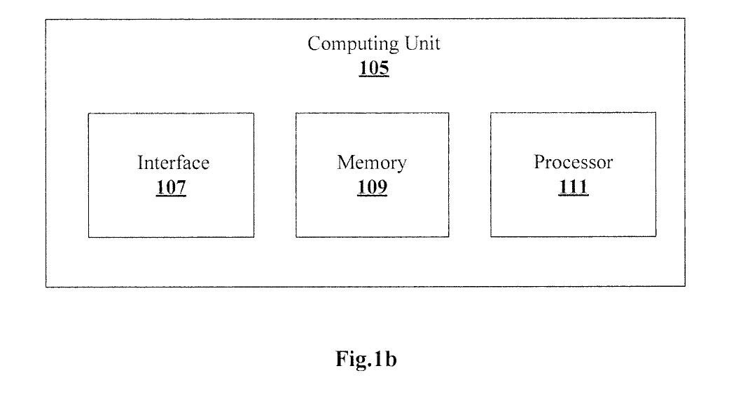

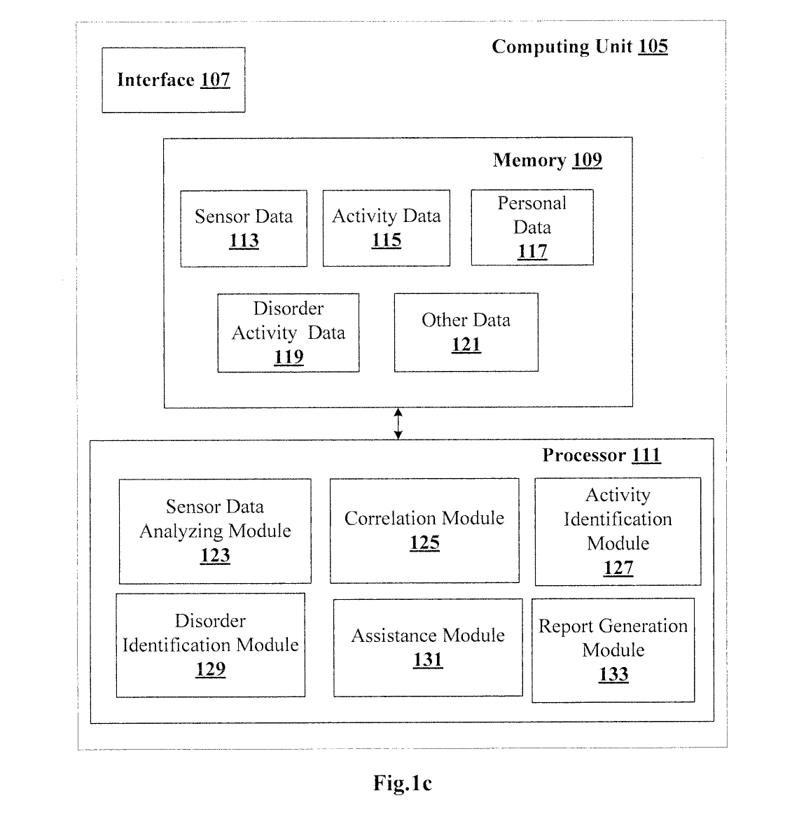

FIG. 1a illustrates an environment for determining psychological disorder condition of a person and providing assistance to a person with psychological disorder in accordance with some embodiments of the present disclosure.

As shown in FIG. 1a, the environment 100 may include one or more sensors, S1 101₁ to SN 101n (collectively referred to as one or more sensors 101) and a computing unit 105. The one or more sensors 101 include, but are not limited to, a camera, an Electromyography (EMG), an accelerometer, a location sensor and a gyroscope. A person skilled in the art would understand that any other sensor capable of capturing vitals of a person can be used with the present disclosure. The one or more sensors 101 are placed on a person under observation to monitor activities of the person. In one implementation, the one or more sensors 101 continuously monitor the activities of the person and provides sensor data associated with the monitored activities to the computing unit 105. Based on the sensor data, the computing unit 105 determines the probability of psychological disorder condition in the person and provides assistance to the person with the psychological disorder. The computing unit 105 includes, but is not limited to, a mobile phone, a tablet and a computer or any other computing device capable of performing the processing required by method of present disclosure. In another implementation, the one or more sensors 101 continuously monitor the activities of the person and provide sensor data associated with the monitored activities to a data aggregation unit (not shown explicitly in FIG. 1a). The one or more sensors communicate with the data aggregation unit using one or more communication protocols which include, but are not limited to, Bluetooth and Zigbee. The data aggregation unit aggregates the sensor data from each of the one or more sensors 101 at one or more time intervals and provides the aggregated data to the computing unit 105.

FIG. 1b illustrates a block diagram of an example of a psychological disorder management computing unit device or computing unit 105 in accordance with some embodiments of the present disclosure.

As shown in FIG. 1b, the computing unit 105 may include an interface 107, a memory 109 and a processor 111. The interface 107 is coupled with the processor 111. The sensor data are received from the one or more sensors 101 through the interface 107. The memory 109 is communicatively coupled to the processor 111. The memory 109 stores processor-executable instructions which are executable by the processor 111. The instructions configure the processor 111 to determine psychological condition in a person and to provide assistance to a person with the psychological disorder condition.

FIG. 1c illustrates a detailed block diagram of a computing unit 105 in accordance with some embodiments of the present disclosure.

As shown in FIG. 1c, the interface 107 is an I/O interface. Using the I/O interface, the computing unit 105 may communicate with one or more I/O devices. The computing unit 105 receives the sensor data from the one or more sensors 101 through the interface 107. In an embodiment, the memory 109 may include sensor data 113, activity data 115, personal data 117, disorder activity data 119 and other data 121.

In one implementation, the sensor data 113 includes information of one or more activities of the person, information of location of the person and information associated with environment of the person while performing the one or more activities. The one or more activities may include, but are not limited to, washing hands, locking door and switching off lights. As an example, the sensor S1 101₁ may be a camera and the sensor S2 101₂ may be an EMG. The activity performed by the person may be “locking the door”. S1 101₁ records the surrounding/environment around the person while locking the door and S2 101₂ records different hand movements of the person while locking the door. Thus, the sensor data 113 from S1 101₁ corresponds to surrounding around the person while locking the door and the sensor data 113 from S2 101₂ corresponds to different hand movements of the person while locking the door.

The activity data 115 may include information of one or more activities. The one or more activities may include, but are not limited to, washing hands, locking door and switching off lights. The personal data 117 may include information of the person. The information of the person may include, but are not limited to, name, qualification, profession, health condition and daily routines of the person.

The disorder activity data 119 may include information of one or more disorder activities provided by the user. The one or more disorder activities include, but are not limited to, washing hands repeatedly, checking for door lock repeatedly and repeating certain words. The memory 109 may also include other data 121 which may comprise temporary data and temporary files, generated by the processor 111 for performing the various functions of the computing unit 105.

In an embodiment, the data in the memory 109 are processed by modules of the computing unit 105. The modules may be stored within the memory 109. As used herein, the term module refers to an application specific integrated circuit (ASIC), an electronic circuit, a processor (shared, dedicated, or group) and memory that execute one or more software or firmware programs, a combinational logic circuit, and/or other suitable components that provide the described functionality. The modules may include, for example, a sensor data analyzing module 123, a correlation module 125, an activity identification module 127, a disorder identification module 129, an assistance module 131 and report generation module 133.

In an embodiment, the sensor data analyzing module 123 processes the sensor data 113 received from each of the one or more sensors 101. The one or more sensors 101 may continuously monitor the activities of the person and provide the sensor data 113 associated with the monitored activities to the computing unit 105. The sensor data 113 received by the computing unit 105 is not in a form which may be processed by the computing unit. Therefore, the sensor data analyzing module 123 processes the sensor data 113 at each of one or more time intervals to obtain processed sensor data. The processed sensor data may include one or more information such as information associated with one or more objects in the activity performed by the person, information associated with location of the person and information associated with one or more features of the person while performing the activity. The processed sensor data is provided to the correlation module 125. In an embodiment, the one or more time intervals are set by a user of the computing unit 105 i.e the one or more time intervals are preconfigured in the computing unit 105. As an example, the user of the computing unit 105 may be a physician of the person or care taker of the person.

In an embodiment, the correlation module 125 correlates the processed sensor data at each of the one or more time intervals to generate a correlated data. The correlated data represents the activity. As an example, the one or more information may be information associated with one or more objects in the activity performed by the person, location of the person and the information associated with one or more features of the person while performing the activity. The correlation module 125 correlates each of the one or more information at each of the one or more time interval to generate the correlated data.

In an embodiment, the activity identification module 127 compares the correlated data at each of the one or more time intervals with one or more activity data 115 stored in the memory 109. Each of the activity data 115 is associated with an activity. The activity data 115 with which the correlated data matches is selected and the corresponding activity is identified. The activity identification module 127 identifies the activity performed by the person at each of the one or more time intervals. As an example, at time t1 the person may perform the activity of washing hands. At time t2, the person may switch off the lights, at time t3, the person may again perform the activity of washing hands and at time t4 the person may again perform the activity of washing hands.

The disorder identification module 129 compares the one or more activities obtained at each of the one or more time intervals with each other to identify a recurring activity. In the above example, the recurring activity performed by the person is “washing hands”. The disorder identification module 129 determines a probability value and a weighed ratio for each of one or more probability parameters associated with the recurring activity. In a non-limiting embodiment, the probability parameters are similarity between the one or more activities (Psim), frequency of the recurring activities (Pfrq), one or more non-similar activities performed between the recurring activities (Pnrep) and similarity in environment around the person while performing the recurring activity (Penv). In a non-limiting embodiment, the weighted ratio ranges from 0-1. The disorder identification module 129 identifies the probability of the psychological disorder condition in the person based on the probability value and the weighted ratio of the probability parameters.

The assistance module 131 provides assistance to the person with psychological disorder condition in real-time. The one or more disorder activities of the person are stored in the memory 109. The assistance module 131 compares the one or more activities with one or more disorder activities. If the identified activity matches with the disorder activity, the assistance module 131 provides one or more indication to the person in real-time to assist the person with the psychological disorder condition.

The report generation module 133 generates a report upon identifying the psychological disorder condition in the person. The report contains the information which includes, but is not limited to, recurring activity information and information associated with the frequency between the recurring activities.

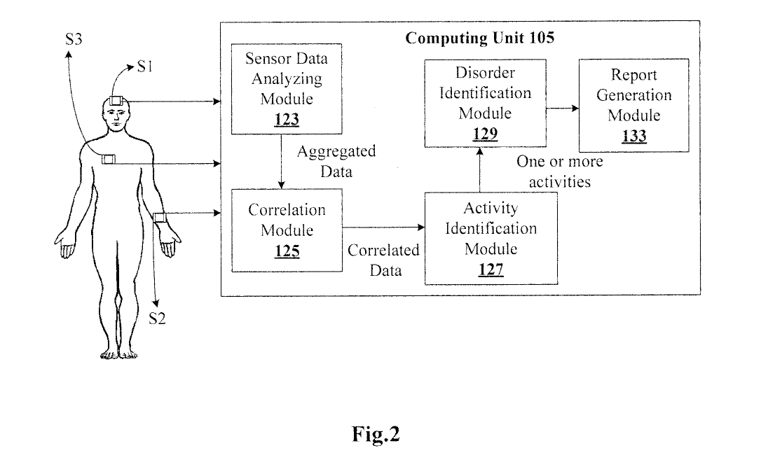

FIG. 2 illustrates an exemplary environment for determining psychological disorder condition in a person in accordance with some embodiments of the present disclosure.

As shown in FIG. 2, the sensors S1 101₁, S2 101₂ and S3 101₃ are placed on the person. S1 is a camera placed on forehead of the person, S2 is an EMG placed on around wrist of the person and S3 is a location sensor placed on chest of the person. The sensors S1 101₁, S2 101₂ and S3 101₃ continuously monitor the activities performed by the person and provide the sensor data 113 associated with the activities performed by the person to the computing unit 105. The sensor data analyzing module 123 processes the sensor data 113 at each of the one or more time intervals configured in the computing unit 105. As an example, the one or more time intervals are t1, t2 and t3. The time interval t1 may be 8 am-8:30 am, the time interval t2 may be 8:30 am-9 am and the time interval t3 may be 9 am-10 am. The sensor data analyzing module 123 processes the sensor data 113 from S1 101₁, S2 101₂ and S3 101₃ at t1, t2 and t3.

In an exemplary embodiment, the activity performed by the person at time interval t1 is “washing hands”. The sensor S1 101₁ monitors the surrounding region/environment around the person while washing the hands. The objects in the environment around the person are “wash basin” and “soap dispenser”. The sensor S2 101₂ measures the movement of the hands while washing the hands. The sensor S3 101₃ monitors the location of the person. The sensor data analyzing module 123 processes the sensor data 113 from the sensors S1 101₁, S2 101₂ and S3 101₃ for obtaining processed sensor data and provides the processed sensor data to the correlation module 125. The correlation module 125 correlates the processed sensor data to generate a correlated data. The correlation module 125 correlates the processed sensor data using machine learning techniques which includes, but are not limited to, neural networks, regression techniques and random forest techniques. The activity identification module 127 compares the correlated data with one or more activity data 115 stored in the memory 109. Upon identifying a match between the correlated data and the activity data 115, the activity corresponding to the activity data 115 is identified.

Similarly, the activity identification module 127 identifies one or more activities associated with the sensor data 113 from the sensors S1 101₁, S2 101₂ and S3 101₃ at time intervals t2 and t3. The activity performed by the person at time interval t2 is “locking the door”. The sensor data 113 from the sensor S1 101₁ is associated with the environment around the person while locking the door. The one or more objects in the environment around the person include a “key”, “key hole” and “door”. The sensor data 113 from the sensor S2 101₂ is associated with movement of the hands while locking the door. The sensor data 113 from the sensor S3 101₃ is associated with the location of the person. The activity performed by the person at time interval t3 is “locking door”. The sensor data from the sensor S1 101₁ is associated with the environment around the person while locking the door. The one or more objects in the environment around the person include a “key”, “key hole” and “door”. The sensor data 113 from the sensor S3 101₃ is associated with the location of the person.

Upon identifying the activities performed by the person at time interval t1 to t3, the disorder identification module 129 compares the activities with each other. The disorder identification module 129 detects the recurring activity among the one or more activities. The recurring activity is “locking door”. The probability parameters associated with the recurring activity are Psim, Pfrq, Pnrep and Penv. The disorder identification module 129 determines the probability value and the weighted ratio for each of the probability parameters. The determined probability values and the weighted ratio for each of the probability parameter is provided in the below Table 1.

TABLE 1

| Probability parameters | Values | Weighted ratio |
|---|---|---|
| Psim | 0.8 | 0.3 |
| Pfrq | 0.6 | 0.1 |
| Penv | 0.8 | 0.1 |
| Pnrep | 0.1 | 0.5 |

The probability value assigned to Psim and Penv is more because there is more similarity in the activity performed by the person and the surroundings of the person while performing the activity. The value assigned to Pfrq is less because the frequency between the recurring activities is not more. The value assigned to Pnrep is less because there are no non-similar activities between the recurring activities.

The disorder identification module 129 identifies the probability of the psychological disorder in the person using the below equation (1).

PPDC=Wt1*Psim+Wt2*Pfrq+Wt3*Penv+Wt4*(1−Pnrep) equation (1)
Wherein: PPDC—Probability of the Psychological disorder condition
Psim—Probability of the similar activities
Pfrq—probability of the frequency between the recurring activities
Penv—Probability of the similar environments while performing the recurring activity
Pnrep—probability of the one or more non-similar activities performed between the recurring activities.

$$\begin{matrix}P P D C = Wt {1}^{*} P s i m + Wt {2}^{*} P f r q + Wt {3}^{*} P e n v + Wt {4}^{*} ( 1 - P n r e p ) \\ = {0.3}^{*} 0.8 + {0.1}^{*} 0.6 + {0.1 }^{*} 0.8 + {0.5}^{*} ( 1 - 0.1 ) \\ = 0.24 + 0.06 + 0.08 + 0.45 \\ = 0.83\end{matrix}$$

In an exemplary embodiment, the person being monitored is a physician. The recurring activity performed by the physician is “washing hands”. The probability value assigned to Psim is more as there is similarity in the activity performed by the physician. The probability value assigned to Pfrq is more as the frequency between the recurring activities performed by the physician is more. The probability value assigned to Penv is less as the surrounding around the physician may be different while performing the activity of washing hands i.e at one time interval the physician may be washing hands at his room and in other time interval the physician may be washing hands in the patients room. The probability parameter Pnrep is more as it is necessary for the physician for repeatedly performing the activity of “washing hands” when treating patients.

The values for the probability parameters and the weighted ratio assigned to each of the probability parameter is provided in the below Table 2.

TABLE 2

| Probability parameters | Values | Weighted ratio |
|---|---|---|
| Psim | 0.9 | 0.3 |
| Pfrq | 0.8 | 0.15 |
| Penv | 0.4 | 0.05 |
| Pnrep | 0.8 | 0.5 |

The disorder identification module 129 identifies the probability of the psychological disorder in the person using the equation (1).

$$\begin{matrix}P P D C = Wt {1}^{*} P s i m + Wt {2}^{*} P f r q + Wt {3}^{*} P e n v + Wt {4}^{*} ( 1 - P n r e p ) \\ = {0.3}^{*} 0.9 + {0.15}^{*} 0.7 + {0.05}^{*} 0.3 + 0.5 ( 1 - 0.8 ) \\ = 0.27 + 0.105 + 0.015 + 0.1 \\ = 0.49\end{matrix}$$

Since the value of PPDC is less than 0.5 the probability of the psychological disorder condition in the person is less.

In an embodiment, the report generation module 133 generates a report upon identifying the probability of the psychological disorder condition in the person. The report may include information associated with the activity performed by the person, quality of the activity, necessity for repeating the activity and the duration after which the activity has to be performed.

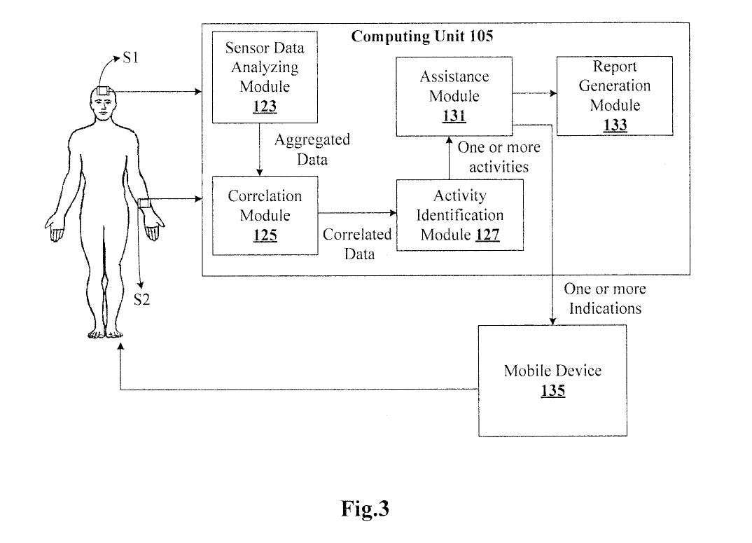

FIG. 3 illustrates an exemplary environment for providing assistance to person with psychological disorder condition in real-time in accordance with some embodiments of the present disclosure.

As shown in FIG. 3, the person is placed with sensors S1 101₁ and S2 101₂. In an exemplary embodiment, the person is aware that he is suffering from OCD. The disorder activity performed by the person repeatedly is “locking the door”. The disorder activity is provided to the computing unit 105. The sensor S1 101₁ is a camera and the sensor S2 101₂ is EMG. The sensors are selected based on the disorder activity provided by the person. In an embodiment, a user of the computing device 105 may provide the one or more time intervals at which the person has to be observed. The sensors S1 101₁ and S2 101₂ monitor the activities of the person continuously and provide the sensor data 113 to the computing unit. The sensor data analyzing module 123 processes the sensor data 113 at each of the one or more time intervals configured in the computing unit 105. As an example, the time intervals are t1, t2 and t3. The sensor data analyzing module 123 analyzes the sensor data 113 from the sensor S1 101₁ and S2 101₂ at t1. The time interval t1 may be 8-8:30 am.

In an exemplary embodiment, the person performs the activity of “locking the door” at time t1. The sensors S1 101₁ and S2 101₂ monitor the activity performed by the person. The sensor S1 101₁ captures the environment around the user while locking the door. The sensor S2 101₂ records the movement of hands while locking the door. The sensor data 113 from the sensors S1 101₁ and S2 101₂ are provided to the computing unit 105. The sensor data analyzing module 123 processes the sensor data 113 at time interval t1 to obtain processed sensor data and provides the processed sensor data to the correlation module 125. The correlation module 125 correlates the processed sensor data to generate a correlated data. The activity identification module 127 compares the correlated data with one or more activity data 115 stored in the memory 109. Upon identifying a match between the correlated data and the one or more activity data 115, the activity corresponding to the one or more activity data 115 is identified. The assistance module 131 compares the identified activity with the list of one or more disorder activities stored in the memory 109. If the identified activity matches with the disorder activity, the assistance module 131 provides one or more indications to assist the person in real-time. The one or more indications may include, but are not limited to, an audio indication, text message or video indication. In the video indication, the video of the activity performed by the person is provided to a mobile device 135 associated with the person. This provides a visual indication to the person that the activity has already been performed. Also, the user may check the quality of the activity being performed. In the audio indication, the computing unit 105 may provide a notification to the mobile device 135. Upon receiving the notification, the mobile device 135 may provide a beep sound to indicate that the activity has already been performed. The indication may also be a text message provided by the computing unit 105 to the mobile device 135.

If the identified activity does not match with the list of one or more disorder activities then the report generation module 133 generates a report. The report may include information associated with the activity performed by the person, quality of the activity, necessity for repeating the activity and the duration after which the activity has to be performed.

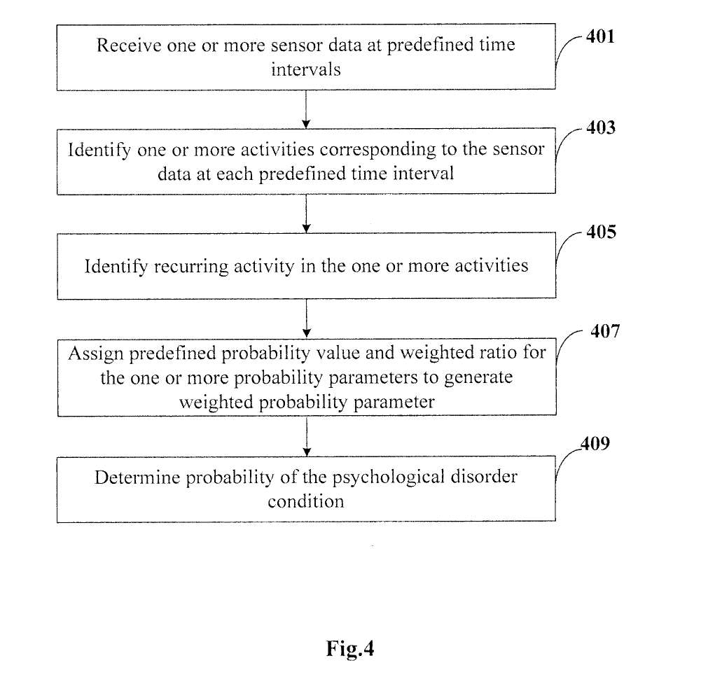

FIG. 4 shows a flowchart illustrating a method for determining psychological disorder condition in a person in accordance with some embodiments of the present disclosure.

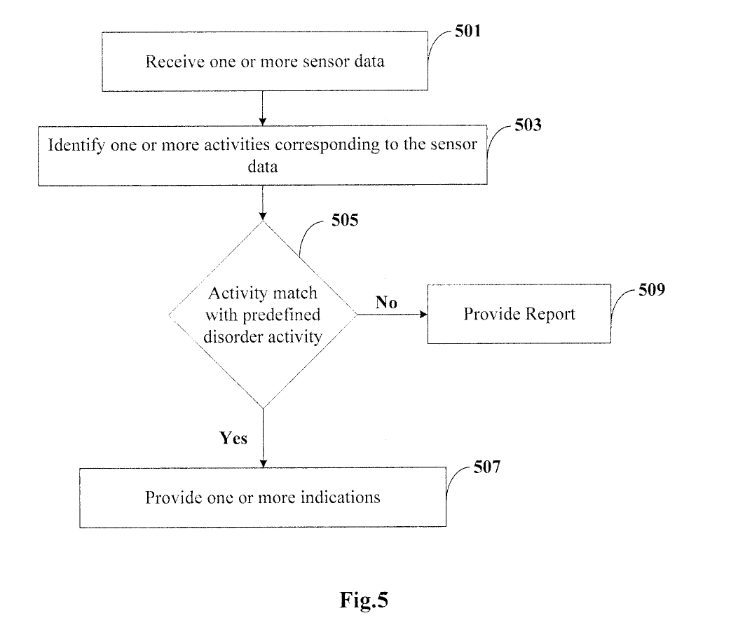

FIG. 5 shows a flowchart illustrating a method for assisting a person with psychological disorder condition in accordance with some embodiments of the present disclosure.

The order in which the methods as described in FIGS. 4-5 is not intended to be construed as a limitation, and any number of the described method blocks can be combined in any order to implement the method. Additionally, individual blocks may be deleted from the method without departing from the spirit and scope of the subject matter described herein. Furthermore, the method can be implemented in any suitable hardware, software, firmware, or combination thereof.

As illustrated in FIG. 4, the method comprises one or more blocks for determining psychological disorder condition in a person. The method may be described in the general context of computer executable instructions. Generally, computer executable instructions can include routines, programs, objects, components, data structures, procedures, modules, and functions, which perform particular functions or implement particular abstract data types.

At block 401, the sensor data 113 are received. In an embodiment, the one or more sensors 101 placed on the person monitors the activities of the person. The computing unit 105 receives the sensor data 113 from the one or more sensors 101 at one or more time intervals. The sensor data analyzing module 123 of the computing unit 105 processes the sensor data 113 at each of the one or more time intervals to obtain processed sensor data and provides the processed sensor data to the correlation module 125. The correlation module 125 correlates the processed sensor data to generate a correlated data.

At block 403, the one or more activities are identified. The activity identification module 127 of the computing unit 105 compares the correlated data with one or more activity data 115 stored in the memory 109. Each of the one or more activity data 115 is associated with a corresponding activity. If the correlated data matches with the one or more activity data 115, the activity corresponding to the matched activity data 115 is selected. Similarly, the one or more activities are identified at one or more time intervals from the sensor data 113.

At block 405, the recurring activity is identified. The disorder identification module 129 of the computing unit 105 compares the one or more activities with each other to identify the recurring activity.

At block 407, the disorder identification module determines a probability value and a weighted ratio for each of one or more probability parameters associated with the recurring activity. The probability value and the weighted ratio are determined to generate a weighted probability parameter.

At block 411, the probability of the psychological disorder condition is determined. The disorder identification module 129 determines the probability of the psychological disorder condition based on each of the weighted probability parameter.

As illustrated in FIG. 5, the method comprises one or more blocks for providing assistance to a person with psychological disorder condition in real-time. The method may be described in the general context of computer executable instructions. Generally, computer executable instructions can include routines, programs, objects, components, data structures, procedures, modules, and functions, which perform particular functions or implement particular abstract data types.

At block 501t, he one or more sensors data are received. In an embodiment, the one or more sensors 101 placed on the person monitors the activities of the person. The computing unit 105 receives the sensor data 113 associated with the monitored activities of the person from the one or more sensors 101. The sensor data analyzing module 123 of the computing unit 105 processes the sensor data 113 at one or more time intervals to obtain a processed sensor data and provides the processed sensor data to the correlation module 125. The correlation module 125 correlates the processed sensor data to generate the correlated data.

At block 503, the one or more activities are identified. The activity identification module 127 of the computing unit 105 compares the correlated data with one or more activity data 115 stored in the memory 109. Each of the one or more activity data 115 is associated with a corresponding activity. If the correlated data matches with the activity data 115, the activity corresponding to the matched activity data 115 is selected.

At block 505, the assistance module 131 of the computing unit 105 compares the one or more activities with the list of one or more disorder activities. If the activity matches with the disorder activity then the method proceeds to block 507 via “Yes”. If the activity does not match with the disorder activity, then the method proceeds to block 509 via “No”.

At block 507, the one or more indications are provided. The assistance module 131 one or more indications may include, but not limited to, an audio indication or video indication. In the video indication, the video of the activity performed by the person is indicated. This provides a visual indication to the person that the activity has already been performed. In the audio indication the computing unit 105 may provide a notification to a mobile device 135 associated with the person. Upon receiving the notification, the mobile device 135 may provide a beep sound to indicate that the activity has already been performed. The notification may also be a text message.

At block 509, the report is generated by the report generation module 133. The report may include information associated with the activity performed by the person, quality of the activity, necessity for repeating the activity and the duration after which the activity has to be performed.

### Computing Unit Device 105

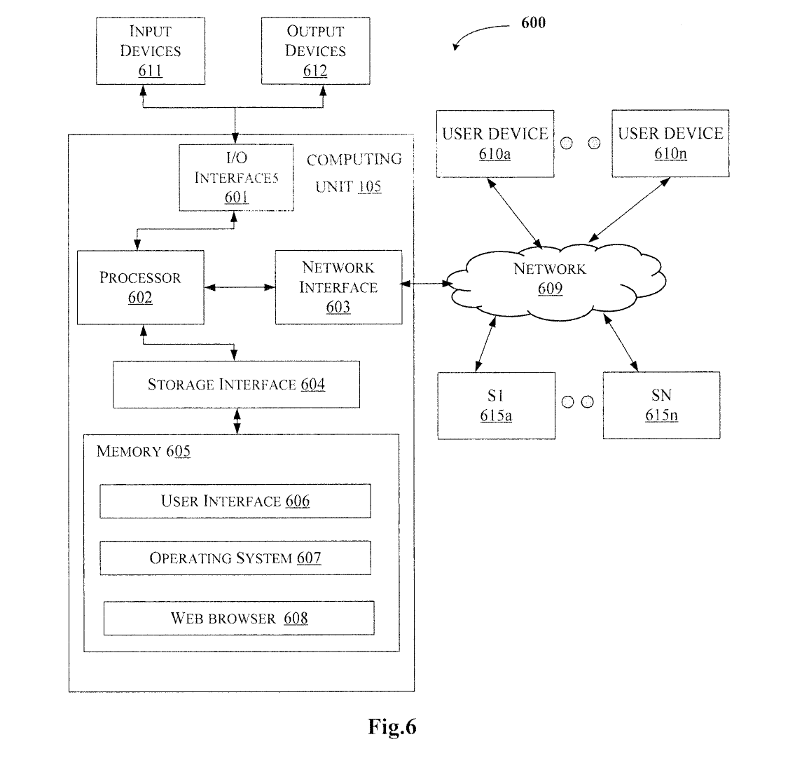

FIG. 6 illustrates a block diagram of an example of a computing unit device or computing unit 105 configured to be capable of implementing embodiments consistent with the present invention as illustrated and described herein. In an embodiment, the computing unit 105 is used to determine psychological disorder condition in a person and to provide assistance to a person with the psychological disorder. The computing unit 105 may comprise a central processing unit (“CPU” or “processor”) 602. The processor 602 may comprise at least one data processor for executing program components for executing user- or system-generated business processes. A user may include a person, a person using a device such as such as those included in this invention, or such a device itself. The processor 602 may include specialized processing units such as integrated system (bus) controllers, memory management control units, floating point units, graphics processing units, digital signal processing units, etc.

The processor 602 may be disposed in communication with one or more input/output (I/O) devices (611 and 612) via I/O interface 601. The I/O interface 601 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, 5-Video, Video Graphics Array (VGA), IEEE 802.n/b/g/n/x, Bluetooth, cellular (e.g., Code-Division Multiple Access (CDMA), High-Speed Packet Access (HSPA+), Global System For Mobile Communications (GSM), Long-Term Evolution (LTE), WiMax, or the like), etc.

Using the I/O interface 601, the computing unit 105 may communicate with one or more I/O devices (611 and 612).

In some embodiments, the processor 602 may be disposed in communication with a communication network 609 via a network interface 603. The network interface 603 may communicate with the communication network 609. The network interface 603 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc. Using the network interface 603 and the communication network 609, the computing unit 105 may communicate with one or more user devices 610 (a, . . . , n) and one or more sensors 615 (a, . . . n). The communication network 609 can be implemented as one of the different types of networks, such as intranet or Local Area Network (LAN) and such within the organization. The communication network 609 may either be a dedicated network or a shared network, which represents an association of the different types of networks that use a variety of protocols, for example, Hypertext Transfer Protocol (HTTP), Transmission Control Protocol/Internet Protocol (TCP/IP), Wireless Application Protocol (WAP), etc., to communicate with each other. Further, the communication network 609 may include a variety of network devices, including routers, bridges, servers, computing devices, storage devices, etc. The one or more user devices 610 (a, . . . , n) may include, without limitation, personal computer(s), mobile devices such as cellular telephones, smartphones, tablet computers, eBook readers, laptop computers, notebooks, gaming consoles, or the like. The one or more sensors 615 (a, . . . n) may include, without limitation, a gyroscope, accelerometer, Electromyography (EMG), camera, or the like.

In some embodiments, the processor 602 may be disposed in communication with a memory 605 (e.g., RAM, ROM, etc. not shown in FIG. 6) via a storage interface 604. The storage interface 604 may connect to memory 605 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etc.

The memory 605 may store a collection of program or database components, including, without limitation, user interface application 606, an operating system 607, web server 608 etc. In some embodiments, computing unit 105 may store user/application data 606, such as the data, variables, records, etc. as described in this invention. Such databases may be implemented as fault-tolerant, relational, scalable, secure databases such as Oracle or Sybase.

The operating system 607 may facilitate resource management and operation of the computing unit 105. Examples of operating systems include, without limitation, Apple Macintosh OS X, UNIX, Unix-like system distributions (e.g., Berkeley Software Distribution (BSD), FreeBSD, NetBSD, OpenBSD, etc.), Linux distributions (e.g., Red Hat, Ubuntu, Kubuntu, etc.), International Business Machines (IBM) OS/2, Microsoft Windows (XP, Vista/7/8, etc.), Apple iOS, Google Android, Blackberry Operating System (OS), or the like. User interface 606 may facilitate display, execution, interaction, manipulation, or operation of program components through textual or graphical facilities. For example, user interfaces may provide computer interaction interface elements on a display system operatively connected to the computing unit 105, such as cursors, icons, check boxes, menus, scrollers, windows, widgets, etc. Graphical User Interfaces (GUIs) may be employed, including, without limitation, Apple Macintosh operating systems' Aqua, IBM OS/2, Microsoft Windows (e.g., Aero, Metro, etc.), Unix X-Windows, web interface libraries (e.g., ActiveX, Java, Javascript, AJAX, HTML, Adobe Flash, etc.), or the like.

In some embodiments, the computing unit 105 may implement a web browser 608 stored program component. The web browser may be a hypertext viewing application, such as Microsoft Internet Explorer, Google Chrome, Mozilla Firefox, Apple Safari, etc. Secure web browsing may be provided using Secure Hypertext Transport Protocol (HTTPS) secure sockets layer (SSL), Transport Layer Security (TLS), etc. Web browsers may utilize facilities such as AJAX, DHTML, Adobe Flash, JavaScript, Java, Application Programming Interfaces (APIs), etc. In some embodiments, the computing unit 105 may implement a mail server stored program component. The mail server may be an Internet mail server such as Microsoft Exchange, or the like. The mail server may utilize facilities such as Active Server Pages (ASP), ActiveX, American National Standards Institute (ANSI) C++/C#, Microsoft .NET, CGI scripts, Java, JavaScript, PERL, PHP, Python, WebObjects, etc. The mail server may utilize communication protocols such as Internet Message Access Protocol (IMAP), Messaging Application Programming Interface (MAPI), Microsoft Exchange, Post Office Protocol (POP), Simple Mail Transfer Protocol (SMTP), or the like. In some embodiments, the computing unit 105 may implement a mail client stored program component. The mail client may be a mail viewing application, such as Apple Mail, Microsoft Entourage, Microsoft Outlook, Mozilla Thunderbird, etc.

Furthermore, one or more non-transitory computer-readable storage media may be utilized in implementing embodiments consistent with the present invention. A non-transitory computer-readable storage medium refers to any type of physical memory on which information or data readable by a processor may be stored. Thus, a non-transitory computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, nonvolatile memory, hard drives, Compact Disc (CD) ROMs, Digital Video Disc (DVDs), flash drives, disks, and any other known physical storage media.

Additionally, advantages of present invention are illustrated herein.

Embodiments of the present disclosure provide a mechanism for determining various symptoms of OCD condition which vary from person to person.

The embodiments of the present disclosure provide a method for providing real-time assistance to the person with the OCD condition.

The present disclosure provides a method for determining the symptoms of OCD without requiring the person to visit the psychiatrist often.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise.

The terms “including”, “comprising”, “having” and variations thereof mean “including but not limited to”, unless expressly specified otherwise.

The enumerated listing of items does not imply that any or all of the items are mutually exclusive, unless expressly specified otherwise.

The terms “a”, “an” and “the” mean “one or more”, unless expressly specified otherwise.

A description of an embodiment with several components in communication with each other does not imply that all such components are required. On the contrary a variety of optional components are described to illustrate the wide variety of possible embodiments of the invention.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method for determining psychological disorder condition in a person, the method comprising:
receiving, by a computing unit, sensor data at predefined time intervals from one or more sensors;
identifying, by the computing unit, one or more activities performed by the person at each predefined time interval based on the sensor data;
identifying, by the computing unit, at least one recurring activity in the one or more activities;
determining, by the computing unit, a probability value and a weighed ratio for each of one or more probability parameters associated with the at least one recurring activity;
generating, by the computing unit, one or more weighted probability parameters based on the probability value and the weighed ratio; and
determining, by the computing unit, probability of the psychological disorder condition in the person based on each of the one or more weighted probability parameters.

2. The method as claimed in claim 1, wherein the one or more sensors are placed on the person.

3. The method as claimed in claim 1, wherein the sensor data is at least one of location of the person, one or more actions performed by the person and one or more objects in the location.

4. The method as claimed in claim 1, wherein the at least one recurring activity is identified by comparing the one or more activities with each other.

5. The method as claimed in claim 1, wherein identifying the one or more activities comprises:
correlating, by the computing unit, each of the sensor data;
comparing, by the computing unit, the correlated data with a list of one or more predefined activity data; and
identifying, by the computing unit, the one or more activities corresponding to the matched one or more predefined activity data.

6. The method as claimed in claim 1, wherein the one or more probability parameters are similarity between the one or more activities, frequency of the recurring activities, one or more non-similar activities performed between the recurring activities and similarity in location of the person while performing the recurring activities.

7. The method as claimed in claim 1 further comprising generating, by the computing unit, a report for providing information associated with the one or more activities and the probability of the psychological disorder condition for each of the one or more activities.

8. A method of providing real-time assistance to a person with psychological disorder condition, the method comprising:
receiving, by a computing unit, sensor data from one or more sensors;
identifying, by the computing unit, one or more activities performed by the person based on the sensor data;
comparing, by the computing unit, the one or more activities with a list of one or more predefined disorder activities;
providing, by the computing unit, one or more indications in real-time for assisting the person with psychological disorder based on the comparison.

9. The method as claimed in claim 8, wherein the one or more sensors are placed on the person.

10. The method as claimed in claim 8, wherein the sensor data is at least one of location of the person, one or more actions performed by the person and one or more objects in the location.

11. The method as claimed in claim 8, wherein identifying the one or more activities comprises:
correlating, by the computing unit, each of the sensor data;
comparing, by the computing unit, the correlated data with a list of one or more predefined activity data; and
identifying, by the computing unit, the one or more activities corresponding to the matched one or more predefined activity data.

12. A computing unit comprising:
a processor; and
a memory communicatively coupled to the processor, wherein the memory stores processor executable instructions, which, on execution, causes the processor to:
receive sensor data at predefined time intervals from one or more sensors placed on the person;
identify one or more activities performed by the person at each predefined time interval based on the sensor data;
identify at least one recurring activity in the one or more activities;
determine a probability value and a weighed ratio for each of one or more probability parameters associated with the at least one recurring activity;
generate one or more weighted probability parameters based on the probability value and the weighed ratio; and
determine probability of the psychological disorder condition in the person based on each of the one or more weighted probability parameters.

13. The computing unit as claimed in claim 12, wherein the instructions configure the at least one processor to identify the one or more activities by performing one or more operations comprising:
correlating each of the sensor data;
comparing the correlated data with a list of one or more predefined activity data; and
identifying the one or more activities corresponding to the matched one or more predefined activity data.

14. The computing unit as claimed in claim 12, wherein the instructions further configure the at least one processor to identify the at least one recurring activity by comparing the one or more activities, identified at each predefined time interval, with each other.

15. The computing unit as claimed in claim 12, wherein the instructions further configure the at least one processor to generate a report for providing information associated with the one or more activities and the probability of the psychological disorder condition for each of the one or more activities.

16. The computing unit as claimed in claim 12, wherein the instructions configure the at least one processor to provide a real-time assistance to the person with psychological disorder by performing one or more operations comprising:
receiving sensor data from one or more sensors;
identifying one or more activities performed by the person based on the sensor data;
comparing the one or more activities with a list of one or more predefined disorder activities; and
providing one or more indications in real-time for assisting the person with psychological disorder based on the comparison.
